SEQUENCE LISTING (29 sequences)

SEQ ID NO: 1 - DNA, 30 bp, Unknown
  oligonucleotide CF101 used in reverse transcriptase PCR
ttttttgaattcgttaccctctctaacttc

SEQ ID NO: 2 - DNA, 33 bp, Unknown
  oligonucleotide FC102 used in reverse transcriptase
tttttttctagattacctccgactgcgtcttga

SEQ ID NO: 3 - DNA, 40 bp, Unknown
  Oligonucleotide BP326 hybridized to modify pVR1020 plasmid
gatctgcagcacgtgtctagaggatatcgaattcgcggcc

SEQ ID NO: 4 - DNA, 40 bp, Unknown
  Oligonucleotide BP329 hybridized to modify pVR1020 plasmid
gatccgcggccgcgaattcgatatcctctagacacgtgct

SEQ ID NO: 5 - DNA, 30 bp, Unknown
  Oligonucleotide FC103 used in reverse transcriptase PCR
ttttttgaattctcactgacagtgcagaca

SEQ ID NO: 6 - DNA, 33 bp, Unknown
  Oligonucleotide FC104 used in reverse transcriptase PCR
tttttttctagattagctgtaagctggggccac

SEQ ID NO: 7 - DNA, 30 bp, Unknown
  Oligonucleotide FC105 used in reverse transcriptase PCR
ttttttgaattcttcaactgccttggaatg

SEQ ID NO: 8 - DNA, 33 bp, Unknown
  Oligonucleotide FC106 used in reverse transcriptase PCR
tttttttctagattaagcgtgcacgttcacgga

SEQ ID NO: 9 - DNA, 36 bp, Unknown
  Oligonucleotide CF107 used in reverse transcriptase PCR
ttttttgatatcaccggaattgcagtcatgattggc

SEQ ID NO: 10 - DNA, 36 bp, Unknown
  Oligonucleotide FC108 used in reverse transcriptase
ttttttgatatcatgtataatgctgatatgattgac

SEQ ID NO: 11 - DNA, 36 bp, Unknown
  Oligonucleotide FC109 used in reverse transcriptase PCR
tttttttctagattaacgttttcccgaggcgaagtc

SEQ ID NO: 12 - DNA, 42 bp, Unknown
  Oligonucleotide C5A1 used in reverse transcriptase PCR
atcatcgagctccagctgtaattcatggtcgaaaagaagtgc

SEQ ID NO: 13 - DNA, 73 bp, Unknown
  Oligonucleotide C5B1 used in reverse transcriptase PCR
gaattcctcgagctgcagcccgggtttttatagctaattagtcattttttgagagtaccacttcagctacctc

SEQ ID NO: 14 - DNA, 72 bp, Unknown
  Oligonucleotide C5C1 used in reverse transcriptase PCR
cccgggctgcagctcgaggaattctttttattgattaactagtcattataaagatctaaaatgcataatttc

SEQ ID NO: 15 - DNA, 45 bp, Unknown
  Oligonucleotide C5D1 used in reverse transcriptase PCR
gatgatggtaccgtaaacaaatataatgaaaagtattctaaacta

SEQ ID NO: 16 - DNA, 34 bp, Unknown
  Oligonucleotide JCA291 used to amplify the vaccine promoter H6
aaacccgggttctttattctatacttaaaaagtg

SEQ ID NO: 17 - DNA, 43 bp, Unknown
  Oligonucleotide JCA292 used to amplify the vaccine promoter H6
aaaagaattcgtcgactacgatacaaacttaacggatatcgcg

SEQ ID NO: 18 - DNA, 33 bp, Unknown
  Oligonucleotide FC110 used in PCR reaction
ttttcgcgaaccggaattgcagtcatgattggc

SEQ ID NO: 19 - DNA, 39 bp, Unknown
  Oligonucleotide FC111 used in PCR reaction
ttttgtcgacgcggccgcttaagcgtgcacgttcacgga

SEQ ID NO: 20 - DNA, 42 bp, Unknown
  Oligonucleotide C6A1 used in PCR reaction
atcatcgagctcgcggccgcctatcaaaagtcttaatgagtt

SEQ ID NO: 21 - DNA, 73 bp, Unknown
  Oligonucleotide C6B1 used in PCR reaction
gaattcctcgagctgcagcccgggtttttatagctaattagtcattttttcgtaagtaagtatttttatttaa

SEQ ID NO: 22 - DNA, 72 bp, Unknown
  Oligonucleotide C6C1 used in PCR reaction
cccgggctgcagctcgaggaattctttttattgattaactagtcaaatgagtatatataattgaaaaagtaa

SEQ ID NO: 23 - DNA, 45 bp, Unknown
  Oligonucleotide C6D1 used in PCR reaction
gatgatggtaccttcataaatacaagtttgattaaacttaagttg

SEQ ID NO: 24 - DNA, 33 bp, Unknown
  Oligonucleotide FC112 used in PCR reaction
aaacccgggcggtggtttgcgattccgaaatct

SEQ ID NO: 25 - DNA, 43 bp, Unknown
  Oligonucleotide FC113 used in PCR reaction
aaaagaattcggatccgattaaacctaaataattgtactttgt

SEQ ID NO: 26 - DNA, 33 bp, Unknown
  Oligonucleotide FC114 used in PCR reaction
tttcacgtgatgtataatgctgatatgattgac

SEQ ID NO: 27 - DNA, 42 bp, Unknown
  Oligonucleotide FC115 used in PCR reaction
ttttggatccgcggccgcttaacgttttcccgaggcgaagtc

SEQ ID NO: 28 - DNA, 39 bp, Unknown
  Oligonucleotide FC116 used in PCR reaction
ttttttgatatcatgaccggaattgcagtcatgattggc

SEQ ID NO: 29 - DNA, 36 bp, Unknown
  Oligonucleotide FC117 used in PCR reaction
ttttcgcgaatgaccggaattgcagtcatgattggc

(19) United States
(12) Patent Application Publication
(10) Pub. No.: US 2004/0241191 A1
(43) Pub. Date: Dec. 2, 2004

(54) Recombinant vaccine against West Nile virus

(76) Inventors: Sheena May Loosmore, Aurora (CA); Jean-Christophe Francis Audonnet, Lyon (FR)

Correspondence Address: FROMMER LAWRENCE & HAUG, 745 FIFTH AVENUE- 10TH FL., NEW YORK, NY, 10151, US

(21) Appl. No.: 10/676,502

(22) Filed: Sep. 30, 2003

Related U.S. Application Data
(63) Continuation of application No. 10/116,298, filed on Apr. 4, 2002, now abandoned.
(60) Provisional application No. 60/281,923, filed on Apr. 6, 2001.

(51) Int. Cl. A61K 39/12

(52) U.S. Cl. 424/204.1

(57) ABSTRACT

Disclosed and claimed are immunogenic compositions to induce an immune response against West Nile (WN) virus, recombinants, for instance recombinant avipox viruses containing and expressing exogenous polynucleotide(s) from WN virus, and methods for making and using the same.

### RELATED APPLICATIONS

This application claims priority from U.S. Provisional application Serial No. 60/281,923, filed Apr. 6, 2001.

### FIELD OF THE INVENTION

The present invention relates to in vivo and in vitro expression vectors comprising and expressing at least one polynucleotide of the West Nile fever virus, as well as immunogenic compositions and vaccines against West Nile fever. It also relates to methods for immunizing and vaccinating against this virus.

Each document cited in this text (*application cited documents*) and each document cited or referenced in each of the application cited documents, is hereby incorporated herein by reference; and, technology in each of the documents incorporated herein by reference can be used in the practice of this invention.

### BACKGROUND OF THE INVENTION

The West Nile fever virus (WNV) was first identified in man in 1937 in Ouganda in the West Nile Province (Zeller H. G., Med. Trop., 1999, 59, 490-494).

Widespread in Africa, it is also encountered in India, Pakistan and the Mediterranean basin and was identified for the first time in the USA in 1999 in New York City (Anderson J. F. et al., Science, 1999, 286, 2331-2333).

The West Nile fever virus affects birds as well as mammals, together with man.

The fever is characterized in birds by an attack of the central nervous system and death. The lesions include encephalitis, hemorrhages in the myocardium and hemorrhages and necroses in the intestinal tract.

In chickens, experimental infections by subcutaneous inoculations of the West Nile fever virus isolated on crows led to necroses of the myocardium, nephrites and pneumonia 5 to 10 days after inoculation and moderate to severe encephalitis 21 days after inoculation (Senne D. A. et al., Avian Disease, 2000, 44, 642-649).

The West Nile fever virus also affects horses, particularly in North Africa and Europe (Cantile C. et al., Equine Vet. J., 2000, 32 (1), 31-35). These horses reveal signs of ataxia, weakness of the rear limbs, paresis evolving towards tetraplegia and death. Horses and camels are the main animals manifesting clinical signs in the form of encephalitis.

Anti-WNV antibodies were detected in certain rodents, in livestock, particularly bovines and ovines, as well as in domestic animals, particularly in the dog (Zeller H. G., Med. Trop., 1999, 59, 490-494; Lundstrom J. O., Journal of Vector Ecology, 1999, 24 (1), 1-39).

The West Nile fever virus also affects with a number of symptoms the human species (Sampson B. A., Human Pathology, 2000, 31 (5), 527-531; Marra C. M., Seminars in Neurology, 2000, 20 (3), 323-327).

The West Nile fever virus is transmitted to birds and mammals by the bites of certain mosquitoes (e.g. Culex, Aedes, Anopheles) and ticks.

Wild and domestic birds are a reservoir for the West Nile virus and a propagation vector as a result of their migrations.

The virions of the West Nile fever virus are spherical particles with a diameter of 50 nm constituted by a lipoproteic envelope surrounding an icosahedric nucleocapsid containing a positive polarity, single-strand RNA.

A single open reading frame (ORF) encodes all the viral proteins in the form of a polyprotein. The cleaving and maturation of this polyprotein leads to the production of about ten different viral proteins. The structural proteins are encoded by the 5′ part of the genome and correspond to the nucleocapsid designated C (14 kDa), the envelope glycoprotein designated E (50 kDa), the pre-membrane protein designated prM (23 kDa), the membrane protein designated M (7 kDa). The non-structural proteins are encoded by the 3′ part of the genome and correspond to the proteins NS1 (40 kDa), NS2A (19 kDa), NS2B (14 kDa), NS3 (74 kDa), NS4A (15 kDa), NS4B (29 kDa), NS5 (97 kDa).

Parrish C. R. et al. (J. Gen. Virol., 1991, 72, 1645-1653), Kulkarni A. B. et al. (J. Virol., 1992, 66 (6), 3583-3592) and Hill A. B. et al. (J. Gen. Virol., 1992, 73, 1115-1123), on the basis of the vaccinia virus, constructed in vivo expression vectors containing various inserts corresponding to nucleotide sequences coding for non-structural proteins of the Kunjin virus, optionally associated with structural proteins. These vectors were administered to the mouse to evaluate the immune cell response. The authors stress the importance of the cell response, which is essentially stimulated by non-structural proteins and especially NS3, NS4A and NS4B. These articles reveal the difficulty in providing a good vaccination strategy against West Nile fever.

Hitherto there is no vaccine preventing infection by the WN virus.

### DESCRIPTION OF THE INVENTION

The present invention relates to a means for preventing and/or combating diseases caused by the WN virus.

Another objective of the invention is to propose such a means usable in different animal species sensitive to the disease caused by said virus and in particular equine and avian species.

Another objective of the invention is to propose immunization and vaccination methods for the target species.

Yet another objective of the invention is to propose means and methods making it possible to ensure a differential diagnosis.

Thus, the first object of the invention is in vitro and/or in vivo expression vectors comprising a polynucleotide encoding the envelope protein E of the WN virus. These vectors also comprise the elements necessary for the expression of the polynucleotide in the host cell.

In addition to the polynucleotide encoding E, the expression vectors according to the invention can comprise one or more other polynucleotides encoding other proteins of the WN virus, preferably structural proteins of the WN virus and said sequences are preferably chosen from among those encoding the pre-membrane protein prM and the membrane protein M.

The vector preferably comprises a polynucleotide forming a single encoding frame corresponding e.g. to prM-E, M-E and more particularly prM-M-E. A vector comprising several separate polynucleotides encoding the different proteins (e.g. prM and/or M and E) also falls within the scope of the present invention. The vector, more particularly in vivo, can also comprise polynucleotides corresponding to more than one WN virus strain, particularly two or more polynucleotides encoding E or prM-M-E of different strains. As will be shown hereinafter, the vector, particularly in vivo, can comprise one or more nucleotide sequences encoding immunogens of other pathogenic agents and/or cytokins.

According to a preferred embodiment of the invention, the expression vector comprises a polynucleotide encoding prM-M-E and preferably in a single reading frame.

The term polynucleotide encoding a protein of the WN virus mainly means a DNA fragment encoding said protein, or the complementary strand of said DNA fragment. An RNA is not excluded.

In the sense of the invention, the term protein covers fragments, including peptides and polypeptides. By definition, the protein fragment is immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises at least one epitope or antigenic determinant. The term epitope relates to a protein site able to induce an immune reaction of the humoral type (B cells) and/or cellular type (T cells).

Thus, the minimum structure of the polynucleotide is that encoding an epitope or antigenic determinant of the protein in question. A polynucleotide encoding a fragment of the total protein more particularly comprises a minimum of 21 nucleotides, particularly at least 42 nucleotides and preferably at least 57, 87 or 150 consecutive nucleotides of the sequence in question. Epitope determination procedures are well known to the one skilled in the art and it is more particularly possible to use overlapping peptide libraries (Hemmer B. et al., Immunology Today, 1998, 19 (4), 163-168), Pepscan (Geysen H. M. et al., Proc. Nat. Acad. Sci. USA, 1984, 81 (13), 3998-4002; Geysen H. M. et al., Proc. Nat. Acad. Sci. USA, 1985, 82 (1), 178-182; Van der Zee R. et al., Eur. J. Immunol., 1989, 19 (1), 43-47; Geysen H. M., Southeast Asian J. Trop. Med. Public Health, 1990, 21 (4), 523-533; Multipin® Peptide Synthesis Kits de Chiron) and algorithms (De Groot A. et al., Nature Biotechnology, 1999, 17, 533-561).

In particular the polynucleotides according to the invention comprise the nucleotide sequence encoding one or two transmembrane domains and preferably two of them, located in the terminal part C of the protein E. For the WNV NY99 strain, these domains correspond to amino acid sequences 742 to 766 and 770 to 791 of GenBank AF196835.

Elements necessary for the expression of the polynucleotide or polynucleotides are present. In minimum manner, this consists of an initiation codon (ATG), a stop codon and a promoter, as well as a polyadenylation sequence for the plasmids and viral vectors other than poxviruses. When the polynucleotide encodes a polyprotein fragment, e.g. prM-E, M-E, prM-M-E, an ATG is placed at 5′ of the reading frame and a stop codon is placed at 3′. As will be explained hereinafter, other elements making it possible to control the expression could be present, such as enhancer sequences, stabilizing sequences and signal sequences permitting the secretion of the protein.

The present invention also relates to preparations comprising such expression vectors. It more particularly relates to preparations comprising one or more in vivo expression vectors, comprising and expressing one or more of the above polynucleotides, including that encoding E, in a pharmaceutically acceptable excipient or vehicle.

According to a first embodiment of the invention, the other vector or vectors in the preparation comprise and express one or more other proteins of the WN virus, e.g. prM, M, prM-M.

According to another embodiment, the other vector or vectors in the preparation comprise and express one or more proteins of one or more other WN virus strains. In particular, the preparation comprises at least two vectors expressing, particularly in vivo, polynucleotides of different WN strains encoding the same proteins and/or for different proteins, preferably for the same proteins. This is more particularly a matter of vectors expressing in vivo E or prM-M-E of two, three or more different WN strains. The invention is also directed at mixtures of vectors expressing prM, M, E, prM-M, prM-E or M-E of different strains.

According to yet another embodiment and as will be shown in greater detail hereinafter, the other vector or vectors in the preparation comprise and express one or more cytokins and/or one or more immunogens of one or more other pathogenic agents.

The invention also relates to various combinations of these different embodiments.

The preparations comprising an in vitro or in vivo expression vector comprising and expressing a polynucleotide encoding prM-M-E constitute a preferred embodiment of the invention.

According to a special embodiment of the invention, the in vivo or in vitro expression vectors comprise as the sole polynucleotide or polynucleotides of the WN virus, a polynucleotide encoding the protein E, optionally associated with prM and/or M, preferably encoding prM-M-E and optionally a signal sequence of the WN virus.

According to a special embodiment, one or more of the non-structural proteins NS2A, NS2B and NS3 are expressed jointly with the structural proteins according to the invention, either via the same expression vector, or via their own expression vector. They are preferably expressed together on the basis of a single polynucleotide.

Thus, the invention also relates to an in vivo or in vitro expression vector comprising the polynucleotide encoding NS2A, NS2B, NS3, their combinations and preferably for NS2A-NS2B-NS3. Basically said vector can be one of the above-described vectors comprising a polynucleotide encoding one or more structural proteins, particularly E or prM-M-E. As an alternative, the invention relates to a preparation as described hereinbefore, also incorporating at least one of these vectors expressing a non-structural protein and optionally a pharmaceutically acceptable vehicle or excipient.

In order to implement the expression vectors according to the invention, the one skilled in the art has various strains of the WN virus and the description of the nucleotide sequence of their genome, cf. particularly Savage H. M. et al. (Am. J. Trop. Med. Hyg. 1999, 61 (4), 600-611), table 2, which refers to 24 WN virus strains and gives access references to polynucleotide sequences in GenBank.

Reference can e.g. be made to strain NY99 (GenBank AF196835). In GenBank, for each protein the corresponding DNA sequence is given (nucleotides 466-741 for prM, 742-966 for M, 967-2469 for E, or 466-2469 for prM-M-E, 3526-4218 for NS2A, 4219-4611 for NS2B and 4612-6468 for NS3, or NS2A-NS2B-NS3). By comparison and alignment of the sequences, the determination of a polynucleotide encoding such a protein in another WNV strain is immediate.

It was indicated hereinbefore that polynucleotide was understood to mean the sequence encoding the protein or a fragment or an epitope specific to the WN virus. Moreover, by equivalence, the term polynucleotide also covers the corresponding nucleotide sequences of the different WN virus strains and nucleotide sequences differing by the degeneracy of the code.

Within the family of WN viruses, identity between amino acid sequences prM-M-E relative to that of NY99 is equal to or greater than 90%. Thus, the invention covers polynucleotides encoding an amino acid sequence, whose identity with the native amino acid sequence is equal to or greater than 90%, particularly 92%, preferably 95% and more specifically 98%. Fragments of these homologous polynucleotides specific with respect to WN viruses, are also considered equivalents.

Thus, on referring to a polynucleotide of the WN virus, this term covers equivalent sequences within the sense of the invention.

It has also been seen that the term protein covers immunologically active peptides and polypeptides. For the requirements of the invention, it covers:

a) corresponding proteins of the different WN virus strains,

b) proteins differing therefrom, but maintaining with a native WN protein an identity equal to or greater than 90%, particularly 92%, preferably 95% and more specifically 98%.

Thus, on referring to a protein of the WN virus, this term covers equivalent proteins within the sense of the invention.

Different WN virus strains are accessible in collections, particularly in the American Type Culture Collection (ATCC), e.g. under access numbers VR-82 or VR-1267. The Kunjin virus is in fact considered to be a WN virus.

According to the invention, preferably the polynucleotide also comprises a nucleotide sequence encoding a signal peptide, located upstream of the expressed protein in order to ensure the secretion thereof. It can consequently be an endogenic sequence, i.e. the natural signal sequence when it exists (coming from the same WN virus or another strain). For example, for the NY99 WN virus, the endogenic signal sequence of E corresponds to nucleotides 922 to 966 of the GenBank sequence and for prM it is a matter of nucleotides 421 to 465. It can also be a nucleotide sequence encoding a heterologous signal peptide, particularly that encoding the signal peptide of the human tissue plasminogen activator (tPA) (Hartikka J. et al., Human Gene Therapy, 1996, 7, 1205-1217). The nucleotide sequence encoding the signal peptide is inserted in frame and upstream of the sequence encoding E or its combinations, e.g. prM-M-E.

According to a first embodiment of the invention, the in vivo expression vectors are viral vectors.

These expression vectors are advantageously poxviruses, e.g. the vaccinia virus or attenuated mutants of the vaccinia virus, e.g. MVA (Ankara strain) (Stickl H. and Hochstein-Mintzel V., Munch. Med. Wschr., 1971, 113, 1149-1153; Sutter G. et al., Proc. Natl. Acad. Sci. U.S.A., 1992, 89, 10847-10851; commercial strain ATCC VR-1508; MVA being obtained after more than 570 passages of the Ankara vaccine strain on chicken embryo fibroblasts) or NYVAC (its construction being described in U.S. Pat. No. 5,494,807, particularly in examples 1 to 6, said patent also describing the insertion of heterologous genes in sites of this recombinant and the use of matched promoters—reference also to be made to WO-A-96/40241), avipox (in particular canarypox, fowipox, pigeonpox, quailpox), swinepox, raccoonpox and camelpox, adenoviruses, such as avian, canine, porcine, bovine, human adenoviruses and herpes viruses, such as equine herpes virus (EHV serotypes 1 and 4), canine herpes virus (CHV), feline herpes virus (FHV), bovine herpes viruses (BHV serotypes 1 and 4), porcine herpes virus (PRV), Marek's disease virus (MDV serotypes 1 and 2), turkey herpes virus (HVT or MDV serotype 3), and duck herpes virus. When a herpes virus is used, the vector HVT is preferred for the vaccination of the avian species and the vector EHV for the vaccination of horses.

According to one of the preferred embodiments of the invention, the poxvirus expression vector is a canarypox or a fowipox, whereby such poxviruses can possibly be attenuated. Reference can be made to the canarypox commercially available from ATCC under access number VR-111. Attenuated canarypox viruses were described in U.S. Pat. No. 5,756,103 and WO-A-01/05934. Numerous fowlpox virus vaccination strains are available, e.g. the DIFTOSEC CT© strain marketed by MERIAL and the NOBILIS© VARIOLE vaccine marketed by Intervet.

For poxviruses, the one skilled in the art can refer to WO-A-90/12882 and more particularly for the vaccinia virus to U.S. Pat. No. 4,769,330; U.S. Pat. No. 4,722,848; U.S. Pat. No. 4,603,112; U.S. Pat. No. 5,110,587; U.S. Pat. No. 5,494,807; U.S. Pat. No. 5,762,938; for fowlpox to U.S. Pat. No. 5,174,993; U.S. Pat. No. 5,505,941; U.S. Pat. No. 5,766,599; for canarypox to U.S. Pat. No. 5,756,103; for swinepox to U.S. Pat. No. 5,382,425 and for raccoonpox to WO-A-00/03030.

When the expression vector is a vaccinia virus, the insertion sites for the polynucleotide or polynucleotides to be expressed are in particular the gene of thymidine kinase (TK), the gene of hemagglutinin (HA), the region of the inclusion body of the A type (ATI). In the case of canarypox, the insertion sites are more particularly located in or are constituted by ORFs, C3, C5 and C6. In the case of fowlpox, the insertion sites are more particularly located in or constituted by the ORFs F7 and F8.

The insertion of genes in the MVA virus has been described in various publications, including Carroll M. W. et al., Vaccine, 1997, 15 (4), 387-394; Stittelaar K. J. et al., J. Virol., 2000, 74 (9), 4236-4243;Suiter G. et al., 1994, Vaccine, 12 (11),1032-1040, to which the one skilled in the art can refer. The complete MVA genome is described in Antoine G., Virology, 1998, 244, 365-396, which enables the one skilled in the art to use other insertion sites or other promoters.

Preferably, when the expression vector is a poxvirus, the polynucleotide to be expressed is inserted under the control of a specific poxvirus promoter, particularly the vaccine promoter 7.5 kDa (Cochran et al., J. Virology, 1985, 54, 30-35), the vaccine promoter 13L (Riviere et al., J. Virology, 1992, 66, 3424-3434), the vaccine promoter HA (Shida, Virology, 1986, 150, 451-457), the cowpox promoter ATI (Funahashi et al., J. Gen. Virol., 1988, 69, 35-47), or the vaccine promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836).

Preferably, for the vaccination of mammals the expression vector is a canarypox. Preferably, for the vaccination of avians, particularly chickens, ducks, turkeys and geese, the expression vector is a canarypox or a fowipox.

When the expression vector is a herpes virus HVT, appropriate insertion sites are more particularly located in the BamHI I fragment or in the BamHI M fragment of HVT. The HVT BamHI I restriction fragment comprises several open reading frames (ORFs) and three intergene regions and comprises several preferred insertion zones, namely the three intergene regions 1, 2 and 3, which constitute preferred regions, and ORF UL55 (FR-A-2 728 795, U.S. Pat. No. 5,980,906). The HVT BamHI M restriction fragment comprises ORF UL43, which is also a preferred insertion site (FR-A-2 728 794, U.S. Pat. No. 5,733,554).

When the expression vector is an EHV-1 or EHV-4 herpes virus, appropriate insertion sites are in particular TK, UL43 and UL45 (EP-A-668355).

Preferably, when the expression vector is a herpes virus, the polynucleotide to be expressed is inserted under the control of a strong eukaryote promoter, preferably the CMV-IE promoter. These strong promoters are described hereinafter in the part of the description relating to plasmids.

According to a second embodiment of the invention, the in vivo expression vectors are plasmidic vectors known as plasmids.

The term plasmid covers any DNA transcription unit in the form of a polynucleotide sequence comprising a polynucleotide according to the invention and the elements necessary for its in vivo expression. Preferably there is a supercoiled or non-supercoiled, circular plasmid. The linear form also falls within the scope of the invention.

Each plasmid comprises a promoter able to ensure, in the host cells, the expression of the polynucleotide inserted under its dependency. In general, it is a strong eukaryote promoter. The preferred strong eukaryote promoter is the early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. No. 5,168,062, U.S. Pat. No. 5,385,839, U.S. Pat. No. 4,968,615, WO-A-87/03905. Preference is given to human CMV-IE (Boshart M. et al., Cell., 1985, 41, 521-530) or murine CMV-IE.

In more general terms, the promoter has either a viral or a cellular origin. A strong viral promoter other than CMV-IE is the early/late promoter of the SV40 virus or the LTR promoter of the Rous sarcoma virus. A strong cellular promoter is the promoter of a gene of the cytoskeleton, such as e.g. the desmin promoter (Kwissa M. et al., Vaccine, 2000, 18 (22), 2337-2344), or the actin promoter (Miyazaki J. et al., Gene, 1989, 79 (2), 269-277).

By equivalence, the subfragments of these promoters, maintaining an adequate promoting activity are included within the present invention, e.g. truncated CMV-IE promoters according to WO-A-98/00166. The notion of the promoter according to the invention consequently includes derivatives and subfragments maintaining an adequate promoting activity, preferably substantially similar to that of the actual promoter from which they are derived. For CMV-IE, this notion comprises the actual promoter part and/or the enhancer part, as well as derivatives and subfragments.

Preferably, the plasmids comprise other expression control elements. It is in particular advantageous to incorporate stabilizing sequences of the intron type, preferably intron II of the rabbit β-globin gene (van Ooyen et al., Science, 1979, 206: 337-344).

As the polyadenylation signal (polyA) for the plasmids and viral vectors other than poxviruses, use can more particularly be made of the one of the bovine growth hormone (bGH) gene (U.S. Pat. No. 5,122,458), the one of the rabbit β-globin gene or the one of the SV40 virus.

The other expression control elements usable in plasmids can also be used in herpes virus expression vectors.

According to another embodiment of the invention, the expression vectors are expression vectors used for the in vitro expression of proteins in an appropriate cell system. The proteins can be harvested in the culture supernatant after or not after secretion (if there is no secretion a cell lysis is done), optionally concentrated by conventional concentration methods, particularly by ultrafiltration and/or purified by conventional purification means, particularly affinity, ion exchange or gel filtration-type chromatography methods.

Production takes place by the transfection of mammal cells by plasmids, by replication of viral vectors on mammal cells or avian cells, or by Baculovirus replication (U.S. Pat. No. 4,745,051; Vialard J. et al., J. Virol., 1990 64 (1), 37-50; Verne A., Virology, 1988, 167, 56-71), e.g. Autographa californica Nuclear Polyhedrosis Virus AcNPV, on insect cells (e.g. Sf9 Spodoptera frugiperda cells, ATCC CRL 1711). Mammal cells which can be used are in particular hamster cells (e.g. CHO or BHK-21) or monkey cells (e.g. COS or VERO). Thus, the invention also covers expression vectors incorporating a polynucleotide according to the invention, the thus produced WN proteins or fragments and the preparations containing the same.

Thus, the present invention also relates to WN protein-concentrated and/or purified preparations. When the polynucleotide encodes several proteins, they are cleaved, and the aforementioned preparations then contain cleaved proteins.

The present invention also relates to immunogenic compositions and vaccines against the WN virus comprising at least one in vivo expression vector according to the invention and a pharmaceutically acceptable excipient or vehicle and optionally an adjuvant.

The immunogenic composition notion covers any composition which, once administered to the target species, induces an immune response directed against the WN virus. The term vaccine is understood to mean a composition able to induce an effective protection. The target species are equines, canines, felines, bovines, porcines, birds, preferably the horse, dog, cat, pig and in the case of birds geese, turkeys, chickens and ducks and which by definition covers reproducing animals, egg-layers and meat animals.

The pharmaceutically acceptable vehicles or excipients are well known to the one skilled in the art. For example, it can be a 0.9% NaCl saline solution or a phosphate buffer. The pharmaceutically acceptable vehicles or excipients also cover any compound or combination of compounds facilitating the administration of the vector, particularly the transfection, and/or improving preservation.

The doses and dose volumes are defined hereinafter in the general description of immunization and vaccination methods.

The immunogenic compositions and vaccines according to the invention preferably comprise one or more adjuvants, particularly chosen from among conventional adjuvants. Particularly suitable within the scope of the present invention are (1) polymers of acrylic or methacrylic acid, maleic anhydride and alkenyl derivative polymers, (2) immunostimulating sequences (ISS), particularly oligodeoxyribonucleotide sequences having one ore more non-methylated CpG units (Klinman D. M. et al., Proc. Natl. Acad. Sci., USA, 1996, 93, 2879-2883; WO-A1-98/16247), (3) an oil in water emulsion, particularly the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, (5) cytokins or (6) their combinations or mixtures.

The oil in water emulsion (3), which is particularly appropriate for viral vectors, can in particular be based on:

light liquid paraffin oil (European pharmacopoeia type),

isoprenoid oil such as squalane, squalene,

oil resulting from the oligomerization of alkenes, particularly isobutene or decene,

esters of acids or alcohols having a straight-chain alkyl group,

more particularly vegetable oils, ethyl oleate, propylene glycol, di(caprylate/caprate), glycerol tri(caprylate/caprate) and propylene glycol dioleate,

esters of branched, fatty alcohols or acids, particularly isostearic acid esters.

The oil is used in combination with emulsifiers to form the emulsion. The emulsifiers are preferably nonionic surfactants, particularly:

esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated,

polyoxypropylene-polyoxyethylene copolymer blocks, particularly Pluronic©, especially L121.

Among the type (1) adjuvant polymers, preference is given to polymers of crosslinked acrylic or methacrylic acid, particularly crosslinked by polyalkenyl ethers of sugars or polyalcohols. These compounds are known under the name carbomer (Pharmeuropa, vol. 8, no. 2, June 1996). The one skilled in the art can also refer to U.S. Pat. No. 2,909,462, which describes such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name Carbopol© (BF Goodrich, Ohio, USA) are particularly suitable. They are in particular crosslinked by allyl saccharose or by allyl pentaerythritol. Among them particular reference can be made to Carbopol© 974P, 934P and 971 P.

Among the maleic anhydride-alkenyl derivative copolymers, preference is given to EMA© (Monsanto), which are straight-chain or crosslinked ethylene-maleic anhydride copolymers and they are e.g. crosslinked by divinyl ether. Reference can be made to J. Fields et al., Nature 186: 778-780, Jun. 4, 1960.

With regards to their structure, the acrylic or methacrylic acid polymers and EMA© are preferably formed by basic units having the following formula:

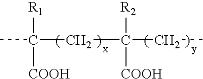

in which:

R₁ and R₂, which can be the same or different, represent H or CH₃

x=0 or 1, preferably x=1

y=1 or2, with x+y=2.

For EMA©, x=0 and y=2 and for carbomers x=y=1.

These polymers are dissolved in water or physiological salt solution (20 g/l NaCl) and the pH is adjusted to 7.3 to 7.4 by soda, in order to give the adjuvant solution in which the expression vectors will be incorporated.

The polymer concentration in the final vaccine composition can range between 0.01 and 1.5% w/v, more particularly 0.05 to 1% w/v and preferably 0.1 to 0.4% w/v.

The cationic lipids (4) containing a quaternary ammonium salt and which are particularly but not exclusively suitable for plasmids, are preferably those complying with the following formula:

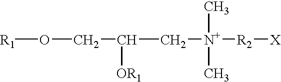

in which R₁ is a saturated or unsaturated straight-chain aliphatic radical having 12 to 18 carbon atoms, R₂ is another aliphatic radical containing 2 or 3 carbon atoms and X is an amine or hydroxyl group.

Among these cationic lipids, preference is given to DMRIE (N-(2-hydroxyethyl)-N,N-dimethyl-2,3-bis(tetradecyloxy)-1-propane ammonium; WO-A-96/34109), preferably associated with a neutral lipid, preferably DOPE (dioleoyl-phosphatidyl-ethanol amine; Behr J. P., 1994, Bioconjugate Chemistry, 5, 382-389) in order to form DMRIE-DOPE.

Preferably, the plasmid mixture with said adjuvant is formed extemporaneously and preferably, prior to its administration, the mixture formed in this way is given time to complex, e.g. for between 10 and 60 minutes and in particular approximately 30 minutes.

When DOPE is present, the DMRIE:DOPE molar ratio is preferably 95:5 to 5:95, more particularly 1:1.

The DMRIE or DMRIE-DOPE adjuvant:plasmid weight ratio is between 50:1 and 1:10, particularly 10:1 and 1:5 and preferably 1:1 and 1:2.

The cytokin or cytokins (5) can be supplied in protein form to the composition or vaccine, or can be co-expressed in the host with the immunogen or immunogens. Preference is given to the co-expression of the cytokin or cytokins, either by the same vector as that expressing the immunogen, or by its own vector.

The cytokins can in particular be chosen from among: interleukin 18 (IL-18), interleukin 12 (IL-12), interleukin 15 (IL-15), MIP-1α (macrophage inflammatory protein 1α; Marshall E. et al., Br. J. Cancer, 1997, 75 (12), 1715-1720), GM-CSF (Granulocyte-Macrophage Colony-Stimulating Factor). Particular reference is made to avian cytokins, particularly those of the chicken, such as cIL-18 (Schneider K. et al., J. Interferon Cytokine Res., 2000, 20 (10), 879-883), cIL-15 (Xin K. -Q. et al., Vaccine, 1999, 17, 858-866), and equine cytokins, particularly equine GM-CSF (WO-A-00/77210). Preferably, use is made of cytokins of the species to be vaccinated.

WO-A-00/77210 describes the nucleotide sequence and the amino acid sequence corresponding to equine GM-CSF, the in vitro GM-CSF production and the construction of vectors (plasmids and viral vectors) permitting the in vivo equine GM-CSF expression. These proteins, plasmids and viral vectors can be used in immunogenic compositions and equine vaccines according to the invention. For example, use can be made of the plasmid pJP097 described in example 3 of said earlier-dated application or use can be made of the teaching of the latter in order to produce other vectors or for the in vitro production of equine GM-CSF and the incorporation of said vectors or said equine GM-CSF in immunogenic compositions or equine vaccines according to the invention.

The present invention also relates to immunogenic compositions and so-called subunit vaccines, incorporating the protein E and optionally one or more other proteins of the WN virus, particularly prM or M and preferably produced by in vitro expression in the manner described hereinbefore, as well as a pharmaceutically acceptable vehicle or excipient.

The pharmaceutically acceptable vehicles or excipients are known to the one skilled in the art and can e.g. be 0.9% NaCl saline solution or phosphate buffer.

The immunogenic compositions and subunit vaccines according to the invention preferably comprise one or more adjuvants, particularly chosen from among conventional adjuvants. Particularly suitable within the scope of the present invention are (1) an acrylic or methacrylic acid polymer, a maleic anhydride and alkenyl derivative polymer, (2) an immunostimulating sequence (ISS), particularly an oligodeoxyribonucleotide sequence having one or more non-methylated CpG units (Klinman D. M. et al., Proc. Natl. Acad. Sci. USA, 1996, 93, 2879-2883; WO-A1-98/16247), (3) an oil in water emulsion, particularly the emulsion SPT described on p 147 of “Vaccine Design, The Subunit and Adjuvant Approach”, published by M. Powell, M. Newmann, Plenum Press 1995, and the emulsion MF59 described on p 183 of the same work, (4) a water in oil emulsion (EP-A-639 071), (5) saponin, particularly Quil-A, or (6) alumina hydroxide or an equivalent. The different types of adjuvants defined under 1), 2) and 3) have been described in greater detail hereinbefore in connection with the expression vector-based vaccines.

The doses and dose volumes are defined hereinafter in connection with the general description of immunization and vaccination methods.

According to the invention, the vaccination against the WN virus can be combined with other vaccinations within the framework of vaccination programs, in the form of immunization or vaccination kits or in the form of immunogenic compositions and multivalent vaccines, i.e. comprising at least one vaccine component against the WN virus and at least one vaccine component against at least one other pathogenic agent. This also includes the expression by the same expression vector of genes of at least two pathogenic agents, including the WN virus.

The invention also relates to a multivalent immunogenic composition or a multivalent vaccine against the WN virus and against at least one other pathogen of the target species, using the same in vivo expression vector containing and expressing at least one polynucleotide of the WN virus according to the invention and at least one polynucleotide expressing an immunogen of another pathogen.

The thus expressed “immunogen” is understood to mean a protein, glycoprotein, polypeptide, peptide, epitope or derivative, e.g. fusion protein, inducing an immune response, preferably of a protective nature.

As was stated hereinbefore, these multivalent compositions or vaccines also comprise a pharmaceutically acceptable vehicle or excipient, and optionally an adjuvant.

The invention also relates to a multivalent immunogenic composition or a multivalent vaccine comprising at least one in vivo expression vector in which at least one polynucleotide of the WN virus is inserted and at least a second expression vector in which a polynucleotide encoding an immunogen of another pathogenic agent is inserted. As stated before, those multivalent compositions or vaccines also comprise a pharmaceutically acceptable vehicle or excipient, and optionally an adjuvant.

For the immunogenic compositions and multivalent vaccines, the other equine pathogens are more particularly chosen from among the group including viruses of equine rhinopneumonia EHV-1 and/or EHV-4 (and preferably there is a combination of immunogens of EHV-1 and EHV-4), equine influenza virus EIV, eastern encephalitis virus EEV, western encephalitis virus WEV, Venezuelan encephalitis virus VEV (preference is given to a combination of the three EEV, WEV and VEV), Clostridium tetani (tetanus) and their mixtures. Preferably, for EHV a choice is made of the genes gB and/or gD; for EIV the genes HA, NP and/or N; for viruses of encephalitis C and/or E2; and for Clostridium tetani the gene encoding all or part of the subunit C of the tetanic toxin. This includes the use of polynucleotides encoding an immunologically active fragment or an epitope of said immunogen.

The other avian pathogens are more particularly chosen from among the group including viruses of the Marek's disease virus MDV (serotypes 1 and 2, preferably 1), Newcastle disease virus NDV, Gumboro disease virus IBDV, infectious bronchitis virus IBV, infectious anaemia virus CAV, infectious laryngotracheitis virus ILTV, encephalomyelitis virus AEV (or avian leukosis virus ALV), virus of hemorragic enteritis of turkeys (HEV), pneumovirosis virus (TRTV), fowl plague virus (avian influenza), chicken hydropericarditis virus, avian reoviruses, Escherichia coli, Mycoplasma gallinarum, Mycoplasma gallisepticum, Haemophilus avium, Pasteurella gallinarum, Pasteurella mulfocida gallicida, and mixtures thereof. Preferably, for MDV a choice is made of the genes gB and/or gD, for NDV the genes HN and/or F; for IBDV the gene VP2; for IBV the genes S (more particularly S1), M and/or N; for CAV the genes VP1 and/or VP2; for ILTV the genes gB and/or gD; for AEV the genes env and/or gag/pro; for HEV the genes 100K and hexon; for TRTV the genes F and/or G and for fowl plague the genes HA, N and/or NP. This includes the use of polynucleotides encoding an immunologically active fragment or an epitope of said immunogen.

By way of example, in a multivalent immunogenic composition or a multivalent vaccine according to the invention, to which an adjuvant has optionally been added in the manner described hereinbefore and which is intended for the equine species, it is possible to incorporate one or more of the plasmids described in WO-A-98/03198 and particularly in examples 8 to 25 thereof, and those described in WO-A-00/77043 and which relate to the equine species, particularly those described in examples 6 and 7 thereof. For the avian species, it is e.g. possible to incorporate one or more of the plasmids described in WO-A1-98/03659, particularly in examples 7 to 27 thereof.

The immunogenic compositions or recombinant vaccines as described hereinbefore can also be combined with at least one conventional vaccine (inactivated, live attenuated, subunits) directed against at least one other pathogen.

In the same way, the immunogenic compositions and subunit vaccines according to the invention can form the object of combined vaccination. Thus, the invention also relates to multivalent immunogenic compositions and multivalent vaccines comprising one or more proteins according to the invention and one or more immunogens (the term immunogen having been defined hereinbefore) of at least one other pathogenic agent (particularly from among the above list) and/or another pathogenic agent in inactivated or attenuated form. In the manner described hereinbefore, these multivalent vaccines or compositions also incorporate a pharmaceutically acceptable vehicle or excipient and optionally an adjuvant.

The present invention also relates to methods for the immunization and vaccination of the target species referred to hereinbefore.

These methods comprise the administration of an effective quantity of an immunogenic composition or vaccine according to the invention. This administration can more particularly take place by the parenteral route, e.g. by subcutaneous, intradermic or intramuscular administration, or by oral and/or nasal routes. One or more administrations can take place, particularly two administrations.

The different vaccines can be injected by a needleless, liquid jet injector. For plasmids it is also possible to use gold particles coated with plasmid and ejected in such a way as to penetrate the cells of the skin of the subject to be immunized (Tang et al., Nature 1992, 356, 152-154).

The immunogenic compositions and vaccines according to the invention comprise an effective expression vector or polypeptide quantity.

In the case of immunogenic compositions or vaccines based on plasmid, a dose consists in general terms about in 10 μg to about 2000 μg, particularly about 50 μg to about 1000 μg. The dose volumes can be between 0.1 and 2 ml, preferably between 0.2 and 1 ml.

These doses and dose volumes are suitable for the vaccination of equines and mammals.

For the vaccination of the avian species, a dose is more particularly between about 10 μg and about 500 μg and preferably between about 50 μg and about 200 μg. The dose volumes can in particular be between 0.1 and 1 ml, preferably between 0.2 and 0.5 ml.

The one skilled in the art has the necessary skill to optimize the effective plasmid dose to be used for each immunization or vaccination protocol and for defining the optimum administration route.

In the case of immunogenic compositions or vaccines based on poxviruses, a dose is in general terms between about 10² pfu and about 10⁹ pfu.

For the equine species and mammals, when the vector is the vaccinia virus, the dose is more particularly between about 10⁴ pfu and about 10⁹ pfu, preferably between about 10⁶ pfu and about 10⁸ pfu and when the vector is the canarypox virus, the dose is more particularly between about 10⁵ pfu and about 10⁹ pfu and preferably between about 10^{5.5} pfu or 10⁶ pfu and about 10⁸ pfu.

For the avian species, when the vector is the canarypox virus, the dose is more particularly between about 10³ pfu and about 10⁷ pfu, preferably between about 10⁴ pfu and about 10⁶ pfu and when the vector is the fowlpox virus, the dose is more particularly between about 10² pfu and about 10⁵ pfu, preferably between about 10³ pfu and about 10⁵ pfu.

In the case of immunogenic compositions or vaccines based on the viral vector other than poxviruses, particularly herpes viruses, a dose is generally between about 10³ pfu and about 10⁸ pfu. In the case of immunogenic compositions or avian vaccines a dose is generally between about 10³ pfu and about 10⁶ pfu. In the case of immunogenic compositions or equine vaccines a dose is generally between about 10⁶ pfu and about 10⁸ pfu.

The dose volumes of the immunogenic compositions and equine vaccines based on viral vectors are generally between 0.5 and 2.0 ml, preferably between 1.0 and 2.0 ml, preferably 1.0 ml. The dose volumes of immunogenic compositions and avian vaccines based on viral vectors are generally between 0.1 and 1.0 ml, preferably between 0.1 and 0.5 ml and more particularly between 0.2 and 0.3 ml. Also in connection with such a vaccine, the one skilled in the art has the necessary competence to optimize the number of administrations, the administration route and the doses to be used for each immunization protocol. In particular, there are two administrations in the horse, e.g. at 35 day intervals.

In the case of immunogenic compositions or subunit vaccines, a dose comprises in general terms about 10 μg to about 2000 μg, particularly about 50 μg to approximately 1000 μg. The dose volumes of the immunogenic compositions and equine vaccines based on viral vectors are generally between 1.0 and 2.0 ml, preferably between 0.5 and 2.0 ml and more particularly 1.0 ml. The dose volumes of the immunogenic compositions and avian vaccines based on viral vectors are generally between 0.1 and 1.0 ml, preferably between 0.1 and 0.5 ml, and more particularly between 0.2 and 0.3 ml. Also for such a vaccine, the one skilled in the art has the necessary skill to optimize the number of administrations, the administration route and the doses to be used for each immunization protocol.

The invention also relates to the use of an in vivo expression vector or a preparation of vectors or polypeptides according to the invention for the preparation of an immunogenic composition or a vaccine intended to protect target species against the WN virus and possibly against at least one other pathogenic agent. The different characteristics indicated in the description are applicable to this object of the invention.

A vaccine based on plasmid or a viral vaccine expressing one or more proteins of the WN virus or a WN subunit vaccine according to the present invention will not induce in the vaccinated animal the production of antibodies against other proteins of said virus, which are not represented in the immunogenic composition or vaccine. This feature can be used for the development of differential diagnostic methods making it possible to make a distinction between animals infected by the WN pathogenic virus and animals vaccinated with vaccines according to the invention. In the former, these proteins and/or antibodies directed against them are present and can be detected by an antigen-antibody reaction. This is not the case with the animals vaccinated according to the invention, which remain negative. In order to bring about this discrimination, use is made of a protein which is not represented in the vaccine (not present or not expressed), e.g. protein C or protein NS1, NS2A, NS2B or NS3 when it is not represented in the vaccine.

Thus, the present invention relates to the use of vectors, preparations and polypeptides according to the invention for the preparation of immunogenic compositions and vaccines making it possible to discriminate between vaccinated animals and infected animals.

It also relates to an immunization and vaccination method associated with a diagnostic method permitting such a discrimination.

The protein selected for the diagnosis or one of its fragments or epitopes is used as the antigen in the diagnostic test and/or is used for producing polyclonal or monoclonal antibodies. The one skilled in the art has sufficient practical knowledge to produce these antibodies and to implement antigens and/or antibodies in conventional diagnostic methods, e.g. ELISA tests.

The invention will now be described in greater detail using embodiments considered as non-limitative examples.

### EXAMPLES

All the constructions are implemented using standard molecular biology methods (cloning, digestion by restriction enzymes, synthesis of a complementary single-strand DNA, polymerase chain reaction, elongation of an oligonucleotide by DNA polymerase . . . ) described by Sambrook J. et al. (Molecular Cloning: A Laboratory Manual, 2nd edition, Cold Spring Harbor Laboratory, Cold Spring Harbor. N.Y., 1989). All the restriction fragments used for the present invention, as well as the various polymerase chain reaction (PCR) are isolated and purified using the Geneclean© kit (B1O101 Inc. La Jolla, Calif.).

### Example 1

Culture of the West Nile Fever Virus

For their amplification, West Nile fever viruses NY99 (Lanciotti R. S. et al., Science, 1999, 286, 2333-7)) are cultured on VERO cells (monkey renal cells), obtainable from the American Type Culture Collection (ATCC) under no. CCL-81.

The VERO cells are cultured in 25 cm² Falcon with eagle-MEM medium supplemented by 1% yeast extracts and 10% calf serum containing approximately 100,000 cells/ml. The cells are cultured at +37° C. under a 5% CO₂ atmosphere.

After three days the cellular layer reaches to confluence. The culture medium is then replaced by the eagle-MEM medium supplemented by 1% yeast extracts and 0.1% cattle serum albumin and the West Nile fever virus is added at a rate of 5 pfu/cell.

When the cytopathogenic effect (CPE) is complete (generally 48 to 72 hours after the start of culturing), the viral suspensions are harvested and then clarified by centrifugation and frozen at −700° C. In general, three to four successive passages are necessary for producing a viral batch, which is stored at −70° C.

### Example 2

Extraction of Viral RNA from the West Nile Fever Virus

The viral RNA contained in 100 ml of viral suspension of the West Nile fever virus strain NY99 is extracted after thawing with solutions of the High Pure Viral RNA Kit Cat # 1 858 882, Roche Molecular Biochemicals, whilst following the instructions of the supplier for the extraction stages. The RNA sediment obtained at the end of extraction is resuspended with 1 to 2 ml of RNase-free, sterile distilled water.

### Example 3

Construction of Plasmid pFC 101

The complementary DNA (ADNC) of the West Nile fever virus NY99 is synthesized with the Gene Amp RNA PCR Kit (Cat # N 808 0017, Perkin-Elmer, Norwalk, Conn. 06859, USA) using the conditions supplied by the manufacture.

A reverse transcriptase polymerase chain reaction (RT-PCR reaction) is carried out with 50 μl of viral RNA suspension of the West Nile fever virus NY99 (example 2) and with the following oligonucleotides:

| CF101 (30 mer) | (SEQ ID NO:1) |
|---|---|
| 5′TTTTTTGAATTCGTTACCCTCTCTAACTTC 3′ | |
| and | |
| FC102 (33 mer) | (SEQ ID NO:2) |
| 5′TTTTTTTCTAGATTACCTCCGACTGCGTCTTGA | |
| 3′ | |

This pair of oligonucleotides allows the incorporation of an EcoRI restriction site, a XbaI restriction site and a stop codon at 3′ of the insert.

The synthesis of the first DNAc strand takes place by elongation of oligonucleotide FC102, following the hybridization of the latter with the RNA matrix.

The synthesis conditions of the first DNAc strand are a temperature of 42° C. for 15 min, then 99° C. for 5 min and finally 4° C. for 5 min. The conditions of the PCR reaction in the presence of the pair of oligonucleotides FC101 and FC102 are a temperature of 95° C. for 2 min, then 35 cycles (95° C. for 1 min, then 62° C. for 1 min and 72° C. for 2 min) and finally 72° C. for 7 min to produce a 302 bp fragment.

This fragment is digested by EcoRI and then by XbaI in order to isolate, following agarose gel electrophoresis, the approximately 290 bp EcoRI-XbaI fragment, which is called fragment A.

The pVR1020 eukaryote expression plasmid (C. J. Luke et al. of Infectious Diseases, 1997, 175, 95-97) derived from the plasmid pVR1012 (FIG. 1 and example 7 of WO-A-98/03199—Hartikka J. et al., 1997, Human Gene Therapy, 7, 1205-1217), contains the frame encoding the signal sequence of the human tissue plasminogen activator (tPA).

A pVR1020 plasmid is modified by BamHI-BglII digestion and insertion of a sequence containing several cloning sites (BamHI, NotI, EcoRI, XbaI, PmlI, PstI, BglII) and resulting from the hybridization of the following oligonucleotides.

BP326 (40 mer)
(SEQ ID NO: 3)
5′GATCTGCAGCACGTGTCTTAGAGGATATCGAATTCGCGGCC 3′
and
BP329 (40 mer)
(SEQ ID No: 4)
5′GATCCGCGGCCGCGAATTCGATATCCTCTAGACACGTGCT 3′

The thus obtained vector with a size of approximately 5105 base pairs (or bp) is called pAB110.

Fragment A is ligatured with the pAB 110 expression plasmid previously digested by XbaI and EcoRI, in order to give the plasmid pFC101 (5376 bp). Under the control of the early promoter of human cytomegalovirus or hCMV-IE (human Cytomegalovirus Immediate Early), said plasmid contains an insert encoding the signal sequence of the activator of tPA followed by the sequence encoding the protein prM.

### Example 4

Construction of Plasmid pFC102

The complementary DNA (DNAc) of the West Nile fever virus NY99 is synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) takes place with 50 μl of viral RNA suspension of the West Nile fever virus NY99 (example 2) and with the following oligonucleotides:

| FC103 (30 mer) | (SEQ ID NO: 5) |
|---|---|
| 5′TTTTTTGAATTCTCACTGACAGTGCAGACA 3′ | |
| and | |
| FC104 (33 mer) | (SEQ ID NO: 6) |
| 5′TTTTTTTCTAGATTAGCTGTAAGCTGGGGCCAC | |
| 3′ | |

This pair of oligonucleotides allows the incorporation of an EcoRI restriction site and a XbaI restriction site and a stop codon at 3′ of the insert.

The first DNAc strand is synthesized by elongation of oligonucleotide FC104, following the hybridization of the latter on the RNA matrix.

The synthesis conditions of the first DNAc strand are a temperature of 42° C. for 15 min, then 99° C. for 5 min and finally 4° C. for 5 min. The conditions of the PCR reaction in the presence of the pair of oligonucleotides FC103 and FC104 are a temperature of 95° C. for 2 min, then 35 cycles (95° C. for 1 min, then 62° C. for 1 min and 72° C. for 2 min) and finally 72° C. for 7 min to produce a 252 bp fragment.

This fragment is digested by EcoRI and then XbaI in order to isolate, following agarose gel electrophoresis, the approximately 240 bp EcoRI-XbaI fragment. This fragment is ligatured with the pAB110 expression plasmid (example 3) previously digested by XbaI and EcoRI in order to give the plasmid pFC102 (5326 bp). Under the control of the early human cytomegalovirus or hCMV-IE (human Cytomegalovirus Immediate Early) promoter, this plasmid contains an insert encoding the signal sequence of the activator of tPA, followed by the sequence encoding the protein M.

### Example 5

Construction of Plasmid pFC103

The complementary DNA (DNAc) of the West Nile fever virus NY99 is synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) takes place with 50 μl of viral RNA suspension of the West Nile fever virus NY99 (example 2) and with the following oligonucleotides:

| FC105 (30 mer) | (SEQ ID NO: 7) |
|---|---|
| 5′TTTTTTGAATTCTTCAACTGCCTTGGAATG 3′ | |
| and | |
| FC106 (33 mer) | (SEQ ID NO: 8) |
| 5′TTTTTTTCTAGATTAAGCGTGCACGTTCACGGA | |
| 3′. | |

This pair of oligonucleotides allows the incorporation of an EcoRI restriction site and a XbaI restriction site, together with a stop codon at 3′ of the insert.

The synthesis of the first DNAc strand takes place by elongation of oligonucleotide FC106, following its hybridization with the RNA matrix.

The synthesis conditions of the first DNAc strand are a temperature of 42° C. for 15 min, then 99° C. for 5 m and finally 4° C. for 5 min. The PCR reaction conditions in the presence of the pair of oligonucleotides FC105 and FC106 are a temperature of 95° C. for 2 min, then 35 cycles (95° C. for 1 min, then 62° C. for 1 min and 72° C. for 2 min), and finally 72° C. for 7 min for producing a 1530 bp fragment.

This fragment is digested by EcoRI and then by XbaI in order to isolate, following agarose gel electrophoresis, the approximately 1518 bp EcorRI-XbaI fragment. This fragment is ligatured with the pAB 110 expression plasmid (example 3) previously digested by XbaI and EcoRI in order to give the plasmid pFC103 (6604 bp). Under the control of the early promoter of human cytomegalovirus or hCMV-IE (human Cytomegalovirus Immediate Early), said plasmid contains an insert encoding the signal sequence of the activator of tPA, followed by the sequence encoding the protein E.

### Example 6

Construction of Plasmid pFC104

The complementary DNA (DNAC) of the West Nile fever virus NY99 is synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) takes place with 50 μl of viral RNA suspension of the West Nile fever virus NY99 (example 2) and with the following oligonucleotides:

FC101 (30 mer) (SEQ ID NO:1)

and FC106 (33 mer) (SEQ ID NO:8)

This pair of oligonucleotides allows the incorporation of an EcoRI restriction site, a XbaI restriction site and a stop codon at 3′ of the insert.

Synthesis of the first DNAC strand takes place by elongation of oligonucleotide FC106, following its hybridization with the RNA matrix.

The synthesis conditions of the first DNAc strand are a temperature of 42° C. for 15 min, then 99° C. for 5 min and finally 4° C. for 5 min. The PCR reaction conditions in the presence of the pair of oligonucleotides FC101 and FC106 are a temperature of 95° C. for 2 min, then 35 cycles (95° C. for 1 min, then 62° C. for 1 min and 72° C. for 2 min) and finally 72° C. for 7 min in order to produce a 2031 bp fragment.

This fragment is digested by EcoRI and then XbaI in order to isolate, following agarose gel electrophoresis, the approximately 2019 bp EcoRI-XbaI fragment. This fragment is ligatured with the pAB110 expression plasmid (example 3), previously digested by XbaI and EcoRI in order to give the pFC104 plasmid (7105 bp). Under the control of the early human cytomegalovirus promoter or hCMV-IE (human Cytomegalovirus Immediate Early), said plasmid contains an insert encoding the signal sequence of the activator of tPA, followed by the sequence encoding the protein prM-M-E.

### Example 7

Construction of Plasmid pFC105

The complementary DNA (DNAc) of the West Nile fever virus NY99 is synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) takes place with 50 μl of viral RNA suspension of the West Nile fever virus NY99 (example 2) and with the following oligonucleotides:

| | |
|---|---|
| CF107 (36 mer) | (SEQ ID NO :9) |
| 5′TTTTTTGATATCACCGGAATTGCAGTCATGATTG | |
| GC 3′ | |
| and | |
| FC106 (33 mer). | (SEQ ID NO :8) |

This pair of oligonucleotides allows the incorporation of an EcoRV restriction site, a XbaI restriction site and a stop codon at 3′ of the insert.

Synthesis of the first DNAc strand takes place by elongation of the FC106 oligonucleotide, following its hybridization with the RNA matrix.

The synthesis conditions of the first DNAc strand are a temperature of 42° C. for 15 min, then 99° C. for 5 min and finally 4° C. for 5 min. The PCR reaction conditions in the presence of the pair of oligonucleotides FC106 and FC107 are a temperature of 95° C. for 2 min, then 35 cycles (95° C. for 1 min, then 62° C. for 1 min and 72° C. for 2 min) and finally 72° C. for 7 min in order to produce a 2076 bp fragment.

This fragment is digested by EcoRV and then XbaI in order to isolate, following agarose gel electrophoresis, the approximately 2058 bp EcoRV-XbaI fragment.

This fragment is ligatured with the pVR1012 expression plasmid, previously digested by XbaI and EcoRV, in order to give the plasmid pFC105 (6953 bp). Under the control of the early human cytomegalovirus promoter or hCMV-IE (human Cytomegalovirus Immediate Early), this plasmid contains an insert encoding the polyprotein prM-M-E.

### Example 8

Construction of Plasmid pFC106

The complementary DNA (DNAc) of the West Nile fever virus NY99 is synthesized with the Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) takes place with 50 μl of viral RNA suspension of the West Nile fever virus NY99 (example 2) and with the following oligonucleotides:

| | |
|---|---|
| FC108 (36 mer) | (SEQ ID NO :10) |
| 5′TTTTTTGATATCATGTATAATGCTGATATGAT | |
| TGAC 3′ | |
| and | |
| FC109 (36 mer) | (SEQ ID NO :11) |
| 5′TTTTTTTCTAGATTAACGTTTTCCCGAGGCGA | |
| AGTC 3′ | |

This pair of oligonucleotides allows the incorporation of an EcoRV restriction site, a XbaI restriction site, an initiating ATG codon in 5′ and a stop codon at 3′ of the insert.

Synthesis of the first DNAc strand takes place by elongation of the oligonucleotide FC109, following its hybridization with the RNA matrix.

The synthesis conditions of the first DNAc strand are a temperature of 42° C. for 15 min, then 99° C. for 5 min and finally 4° C. for 5 min. The PCR reaction conditions in the presence of the pair of nucleotides FC108 and FC109 are a temperature of 95° C. for 2 min, then 35 cycles (95° C. for 1 min, 62° C. for 1 min and then 72° C. for 2 min) and finally 72° C. for 7 min to produce a 2973 bp fragment.

This fragment is digested by EcoRV and then XbaI in order to isolate, following agarose gel electrophoresis, the approximately 2955 bp EcoRV-XbaI fragment.

This fragment is ligatured with the pVR 1012 expression plasmid previously digested by XbaI and EcoRV in order to give the plasmid pFC106 (7850 bp). Under the control of the early human cytomegalovirus promoter or hCMV-IE (human Cytomegalovirus Immediate Early), this plasmid contains an insert encoding the polyprotein NS2A-NS2B-NS3.

### Example 9

Construction of the Donor Plasmid for Insertion in Site C5 of the ALVAC Canarypox Virus

FIG. 16 of U.S. Pat. No. 5,756,103 shows the sequence of a genomic DNA 3199 bp fragment of the canarypox virus. Analysis of this sequence has revealed an open reading frame (ORF) called C5L, which starts at position 1538 and ends at position 1859. The construction of an insertion plasmid leading to the deletion of the ORF C5L and its replacement by a multiple cloning site flanked by transcription and translation stop signals was implemented in the following way.

A PCR reaction was performed on the basis of the matrix constituted by genomic DNA of the canarypox virus and with the following oligonucleotides:

| | |
|---|---|
| C5A1 (42 mer): | (SEQ ID NO :12) |
| 5′ATCATCGAGCTCCAGCTGTAATTCATGGTCGAAAAGAAGTGC 3′ | |
| and | |
| C5B1 (73 mer): | (SEQ ID NO :13) |
| 5′GAATTCCTCGAGCTGCAGCCCGGGTTTTTATAGCTAATTAGTCAT | |
| TTTTTGAGAGTACCACTTCAGCTACCTC 3′ | |

in order to isolate a 223 bp PCR fragment (fragment B).

A PCR reaction was carried out on the basis of the matrix constituted by genomic DNA of the canarypox virus and with the following oligonucleotides:

| | |
|---|---|
| C5C1 (72 mer): | (SEQ ID NO :14) |
| 5′CCCGGGCTGCAGCTCGAGGAATTCTTTTTATTGATTAACTAGTCATTA | |
| TAAAGATCTAAAATGCATAATTTC 3′ | |
| and | |
| C5D1 (45 mer): | (SEQ ID NO :15) |
| 5′GATGATGGTACCGTAAACAAATATAATGAAAAGTATTCTAAACTA 3′ | |

in order to isolate a 482 bp PCR fragment (fragment C).

Fragments B and C were hybridized together in order to serve as a matrix for a PCR reaction performed with the oligonucleotides C5A1 (SEQ ID NO :12) and C5D1 (SEQ ID NO :15) in order to generate a 681 bp PCR fragment. This fragment was digested by the restriction enzymes SacI and KpnI in order to isolate, following agarose gel electrophoresis, a 664 bp SacI-KpnI fragment. This fragment was ligatured with the bplueScript© II SK+vector (Stratagene, La Jolla, USA, Cat # 212205), previously digested by the restriction enzymes SacI and KpnI, in order to give the plasmid pC5L. The sequence of this plasmid was verified by sequencing. This plasmid contains 166 bp of sequences upstream of ORF C5L (left flanking arm C5), an early transcription stop vaccine signal, stop codons in 6 reading frames, a multiple cloning site containing restriction sites SmaI, PstI, XhoI and EcoRI and finally 425 bp of sequences located downstream of ORF C5L (right flanking arm C5).

The plasmid pMP528HRH (Perkus M. et al. J. Virol. 1989, 63, 3829-3836) was used as the matrix for amplifying the complete sequence of the vaccine promoter H6 (GenBank access no. M28351) with the following oligonucleotides:

| | |
|---|---|
| JCA291 (34 mer) | (SEQ ID NO :16) |
| 5′AAACCCGGGTTCTTTATTCTATACTTAAAAAGTG 3′ | |
| and | |
| JCA292 (43 mer) | (SEQ ID NO :17) |
| 5′AAAAGAATTCGTCGACTACGATACAAACTTAACGGATATCGCG 3′ | |

in order to amplify a 149 bp PCR fragment. This fragment was digested by restriction enzymes SmaI and EcoRI in order to isolate, following agarose gel electrophoresis, a 138 bp SmaI-EcoRI restriction fragment. This fragment was then ligatured with the plasmid pC5L, previously digested by SmaI and EcoRI, in order to give the plasmid pFC107.

### Example 10

Construction of the Recombinant Virus vCP1712

A PCR reaction was performed using the plasmid pFC105 (example 7) as the matrix and the following oligonucleotides:

| | |
|---|---|
| FC110 (33 mer: | (SEQ ID NO : 18) |
| 5′TTTTCGCGAACCGGAATTGCAGTCATGATTGGC 3′ | |
| and | |
| FC111 (39 mer): | (SEQ ID NO : 19) |
| 5′TTTTGTCGACGCGGCCGCTTAAGCGTGCACGTTCACGGA 3′ | |

in order to amplify an approximately 2079 bp PCR fragment. This fragment was digested by restriction enzymes NruI and SalI in order to isolate, following agarose gel electrophoresis, an approximately 2068 bp NruI-SalI restriction fragment. This fragment was then ligatured with plasmid pFC107 (example 9) previously digested by restriction enzymes NruI and SalI in order to give the plasmid pFC108.

Plasmid pFC108 was linearized by NotI, then transfected in primary chicken embryo cells infected with the canarypox virus (ALVAC strain) according to the previously described calcium phosphate precipitation method (Panicali et Paoletti Proc. Nat. Acad. Sci. 1982, 79, 4927-4931; Piccini et al. In Methods in Enzymology, 1987, 153, 545-563, publishers Wu R. and Grossman L. Academic Press). Positive plaques were selected on the basis of a hybridization with a radioactively labelled probe specific to the nucleotide sequence of the envelope glycoprotein E. These plaques underwent 4 successive selection/purification cycles until a pure population was isolated. A representative plaque corresponding to in vitro recombination between the donor plasmid pFC108 and the genome of the ALVAC canarypox virus was then amplified and the recombinant virus stock obtained was designated vCP1712.

### Example 11

Construction of the Recombinant Virus vCP1713

Plasmid pFC104 (example 6) was digested by the restriction enzyme SalI and PmlI in order to isolate, following agarose gel electrophoresis, an approximately 2213 bp PmlI-SalI restriction fragment. This fragment was ligatured with plasmid pFC107 (example 9) previously digested by the NruI and SalI restriction enzymes in order to give the plasmid pFC109.

Plasmid pFC109 was linearized by NotI, then transfected in primary chicken embryo cells infected with the canarypox virus (ALVAC strain) according to the method of example 10. A representative plaque corresponding to in vitro recombination between the donor plasmid pFC109 and the genome of the ALVAC canarypox virus was selected on the basis of a hybridization of a radioactively labelled probe specific to the nucleotide sequence of the envelope glycoprotein E and was then amplified. The recombinant virus stock obtained was designated vCP1713.

### Example 12

Construction of the Recombinant Virus vCP1714

Plasmid pFC103 (example 5) was digested by the SalI and PmlI restriction enzymes i order to isolate, following agarose gel electrophoresis, an approximately 1712 bp PmlI-SalI restriction fragment. This fragment was ligatured with the plasmid pFC107 (example 9) previously digested by the NruI and SalI restriction enzymes in order to give the plasmid pFC110.

Plasmid pFC110 was linearized by NotI, then transfected in primary chicken embryo cells infected with the canarypox virus (ALVAC strain) according to the method of example 10. A representative plaque corresponding to in vitro recombination between the donor plasmid pFC110 and the genome of the ALVAC canarypox virus was selected on the basis of a hybridization with a radioactively labelled probe specific to the nucleotide sequence of the envelope glycoprotein E and was then amplified. The recombinant virus stock obtained was then designated vCP1714.

### Example 13

Construction of the Recombinant Virus vCP1715

Plasmid pFC102 (example 4) was digested by the SalI and PmlI restriction enzymes in order to isolate, following agarose gel electrophoresis, an approximately 434 bp PmlI-SalI restriction fragment. This fragment was ligatured with the plasmid pFC107 (example 9) previously digested by the NruI and SalI restriction enzymes to give the plasmid pFC111.

Plasmid pFC111 was linearized by NotI, then transfected in primary chicken embryo cells infected with the canarypox virus (ALVAC strain) according to the method of example 10. A representative plaque corresponding to in vitro recombination between the donor plasmid pFC111 and the genome of the ALVAC canarypox virus was selected on the basis of hybridization with a radioactively labelled probe specific to the nucleotide sequence of the membrane M glycoprotein and was then amplified. The recombinant virus stock obtained was designated vCP1715.

### Example 14

Construction of the Recombinant Virus vCP1716

Plasmid pFC101 (example 3) is digested by the SalI and PmlI restriction enzymes in order to isolate, following agarose gel electrophoresis, an approximately 484 bp PmlI-SalI restriction fragment. This fragment is ligatured with the plasmid pFC107 (example 9) previously digested by the NruI and SalI restriction enzymes to give the plasmid pFC112.

Plasmid pFC112 was linearized by NotI and then transfected in primary chicken embryo cells infected with the canarypox virus (ALVAC strain) according to the method of example 10. A representative plaque corresponding to in vitro recombination between the donor plasmid pFC112 and the genome of the ALVAC canarypox virus was selected on the basis of a hybridization with a radioactively labelled probe specific to the nucleotide sequence of the pre-membrane prM glycoprotein and was then amplified. The recombinant virus stock obtained was designated vCP1716.

### Example 15

Construction of the Donor Plasmid for Insertion in Site C6 of the ALVAC Canarypox Virus

FIG. 4 of WO-A-01/05934 shows the sequence of a 3700 bp genomic DNA fragment of the canarypox virus. Analysis of this sequence revealed an open reading frame (ORF) called C6L, which starts at position 377 and ends at position 2254. The construction of an insertion plasmid leading to the deletion of the ORF C6L and its replacement by a multiple cloning site flanked by transcription and translation stop signals was implemented in the following way.

A PCR reaction was performed on the basis of the matrix constituted by the genomic DNA of the canarypox virus and with the following oligonucleotides:

C6A1 (42 mer) (SEQ ID NO :20):
5′ ATCATCGAGCTCGCGGCCGCCTATCAAAAGTCTTAATGAGTT 3′
and
C6B1 (73 mer) (SEQ ID NO :21):
5′ GAATTCCTCGAGCTGCAGCCCGGGTTTTTATAGCTAATTAGTCATTT
TTTCGTAAGTAAGTATTTTTATTTAA 3′

to isolate a 432 bp PCR fragment (fragment D).

A PCR reaction was performed on the basis of the matrix constituted by the genomic DNA of the canarypox virus and with the following oligonucleotides:

C6C1 (72 mer) (SEQ ID NO :22):
5′ CCCGGGCTGCAGCTCGAGGAATTCTTTTTATTGATTAACTAGTCAAA
TGAGTATATATAATTGAAAAAGTAA 3′
and
C6D1 (45 mer) (SEQ ID NO :23):
5′ GATGATGGTACCTTCATAAATACAAGTTTGATTAAACTTAAG
TTG 3′

to isolate a 1210 bp PCR fragment (fragment E).

Fragments D and E were hybridized together to serve as a matrix for a PCR reaction performed with the oligonucleotides C6A1 (SEQ ID NO:20) and C6D1 (SEQ ID NO:23) to generate a 1630 bp PCR fragment This fragment was digested by the SacI and KpnI restriction enzymes to isolate, after agarose gel electrophoresis, a 1613 bp SacI-KpnI fragment. This fragment was ligatured with the bpluescript© II SK+vector (Stratagene, La Jolla, Calif., USA, Cat # 212205) previously digested by the SacI and KpnI restriction enzymes to give the plasmid pC6L. The sequence of this plasmid was verified by sequencing. Said plasmid contains 370 bp of sequences upstream of ORF C6L (C6 left flanking arm), an early transcription stop vaccinia signal, stop codons in the six reading frames, a multiple cloning site containing the SmaI, PstI, XhoI and EcoRI restriction sites and finally 1156 bp of sequences downstream of the ORF C6L (C6 right flanking arm).

Plasmid pMPIVC (Schmitt J. F. C. et al., J. Virol., 1988, 62, 1889-1897, Saiki R. K. et al., Science, 1988, 239, 487-491) was used as the matrix for amplifying the complete sequence of the I3L vaccine promoter with the following oligonucleotides:

FC112 (33 mer) (SEQ ID NO :24):
5′ AAACCCGGGCGGTGGTTTGCGATTCCGAAATCT 3′
and
FC113 (43 mer) (SEQ ID NO :25):
5′ AAAAGAATTCGGATCCGATTAAACCTAAATAATTGTACTTTGT 3′

to amplify a 151 bp PCR fragment. This fragment was digested by the SmaI and EcoRI restriction enzymes in order to isolate, following agarose gel electrophoresis, an approximately 136 bp SmaI-EcoRI restriction fragment. This fragment was then ligatured with plasmid pC6L previously digested by SmaI and EcoRI to give the plasmid pFC113.

### Example 16

Construction of Recombinant Viruses vCP1717 and vCP1718

A PCR reaction was performed using the plasmid pFC106 (example 8) as the matrix and the following oligonucleotides:

FC114 (33 mer) (SEQ ID NO :26):
5′ TTTCACGTGATGTATAATGCTGATATGATTGAC 3′
and
FC115 (42 mer) (SEQ ID NO :27):
5′ TTTTGGATCCGCGGCCGCTTAACGTTTTCCCGAGGCGAAGTC 3′

to amplify an approximately 2973 bp PCR fragment. This fragment was digested with the PmlI and BamHI restriction enzymes to isolate, following agarose gel electrophoresis, the approximately 2958 bp PmlI-BamHI restriction fragment (fragment F). Plasmid pFC113 (example 15) was digested by the PmlI and BamHI restriction enzymes to isolate, following agarose gel electrophoresis, the approximately 4500 bp PmlI-BamHI restriction fragment (fragment G). Fragments F and G were then ligatured together to give the plasmid pFC114.

Plasmid pFC114 was linearized by NotI, then transfected in primary chicken embryo cells infected with canarypox virus vCP1713 (example 11) according to the previously described calcium phosphate precipitation method (Panicali et Paoletti Proc. Nat. Acad. Sci. 1982, 79, 4927-4931; Piccini et al. In Methods in Enzymology, 1987, 153, 545-563, publishers Wu R. and Grossman L. Academic Press). Positive plaques were selected on the basis of a hybridization with a radioactively labelled probe specific to the nucleotide sequence of envelope glycoprotein E. These ranges underwent four successive selection/purification cycles of the ranges until a pure population was isolated. A representative plaque corresponding to in vitro recombination between the donor plasmid pFC114 and the genome of the ALVAC canarypox virus was then amplified and the recombinant virus stock obtained was designated vCP1717.

The NotI-linearized pFC114 plasmid was also used for transfecting primary chicken embryo cells infected with the vCP1712 canarypox virus (example 10) using the procedure described hereinbefore. The thus obtained recombinant virus stock was designated vCP1718.

### Example 17

Construction of Plasmid pFC115

The complementary DNA (DNAc) of the West Nile fever virus NY99 was synthesized with Gene Amp RNA PCR Kit (Cat # N 808 0017, Perkin-Elmer, Norwalk, Conn. 06859, USA) using the conditions provided by the supplier.

A reverse transcriptase polymerase chain reaction (RT-PCR reaction) was carried out with 50 μl of viral RNA suspension of the West Nile fever virus NY99 (example 2) and with the following oligonucleotides:

FC116 (39 mer) (SEQ ID NO :28)
5′ TTTTTTGATATCATGACCGGAATTGCAGTCATGATTGGC 3′
and
FC106 (33 mer) (SEQ ID NO :8).

This pair of oligonucleotides makes it possible to incorporate an EcoRV restriction site, a XbaI restriction site, an initiator code at 5′ and a stop code at 3′ of the insert.

Synthesis of the first DNAc strand takes place by elongation of the oligonucleotide FC106, following its hybridization with the RNA matrix.

The synthesis conditions of the first DNAc strand are a temperature of 42° C. for 15 min, then 99° C. for 5 min and finally 4° C. for 5 min. The conditions of the PCR reaction in the presence of the pair of oligonucleotides FC106 and FC116 are a temperature of 95° C. for 2 min, then 35 cycles (95° C. for 1 min, 62° C. for 1 min and then 72° C. for 2 min) and finally 72° C. for 7 min to produce a 2079 bp fragment.

This fragment is digested by EcoRV and then XbaI to isolate, following agarose gel electrophoresis, the approximately 2061 bp EcoRV-XbaI fragment.

This fragment is ligatured with the pVR1012 expression plasmid previously digested by XbaI and EcoRV to give the plasmid pFC115 (6956 bp). Under the control of the early human cytomegalovirus promoter or hCMV-IE (human Cytomegalovirus Immediate Early), this plasmid contains an insert encoding the polyprotein prM-M-E.

### Example 18

Construction of the Recombinant Viruses vCP2017

A PCR reaction was carried out using the plasmid pFC115 (example 17) as the matrix and the following oligonucleotides:

FC117 (36 mer) (SEQ ID NO :29):
5′ TTTTCGCGAATGACCGGAATTGCAGTCATGATTGGC 3′
and
FC111 (39 mer) (SEQ ID NO :19)

to amplify an approximately 2082 bp PCR fragment. This fragment was digested by NruI and SalI restriction enzymes to isolate, after agarose gel electrophoresis, an approximately 2071 bp NrI-SalI restriction fragment. This fragment was then ligatured with plasmid pFC107 (example 9) previously digested by the NruI and SalI restriction enzymes to give the plasmid pFC116.

Plasmid pFC116 was linearized by NotI and then transfected in primary chicken embryo cells infected with canarypox virus (ALVAC strain) using the procedure of example 10. A representative plaque corresponding to in vitro recombination between the donor plasmid pFC116 and the genome of the ALVAC canarypox virus was selected on the basis of a hybridization with a radioactively labelled probe specific to the nucleotide sequence of the envelope glycoprotein E and was then amplified. The recombinant virus stock obtained was designed vCP2017.

### Example 19

Production of Recombinant Vaccines

For the preparation of equine vaccines, the recombinant canarypox vCP1712 virus (example 10) is adjuvanted with carbomer solutions, namely Carbopol™974P manufactured by BF Goodrich, Ohio, USA (molecular weight about 3,000,000).

A 1.5% Carbopol™974P stock solution is initially prepared in distilled water containing 1 g/l of sodium chloride. This stock solution is then used for the preparation of a 4 mg/ml Carbopol™974P solution in physiological salt solution. The stock solution is mixed with the adequate volume of said physiological salt solution, either in a single stage or in several successive stages, the pH value being adjusted in each stage with a 1N sodium hydroxide solution (or even more concentrated) in order to obtain a final pH value of 7.3 to 7.4.

The ready-to-use Carbopol™974P solution obtained in this way is used for taking up recombinant, lyophilized viruses or for diluting concentrated, recombinant virus stock solutions. For example, to obtain a viral suspension containing 10⁸ pfu/1 ml dose, a viral stock solution is diluted so as to obtain a titer of 10^{8.3} pfu/ml, followed by dilution in equal parts with said ready-to-use 4 mg/ml Carbopol™974P solution.

Recombinant vaccines can also be produced with recombinant canarypox viruses vCP1713 (example 11) or vCP1717 (example 16) or vCP1718 (example 16) or vCP2017 (example 18) or a mixture of three canarypox viruses vCP1714 (example 12), vCP1715 (example 13) and vCP1716 (example 14) according to the procedure described hereinbefore.

### Example 20

Production of DNA Vaccines for Equines

An DNA solution containing the plasmid pFC104 (example 6) is concentrated by ethanolic precipitation in the manner described by Sambrook et al (1989). The DNA sediment is taken up by a 0.9% NaCl solution so as to obtain a concentration of 1 mg/ml. A 0.75 mM DMRIE-DOPE solution is prepared by taking up a DMRIE-DOPE lyophilizate by a suitable sterile H₂O volume.

The formation of plasmid-lipid DNA complexes is brought about by diluting in equal parts the 0.75 mM DMRIE-DOPE solution (1:1) with the 1 mg/ml DNA solution in 0.9% NaCl. The DNA solution is progressively introduced with the aid of a 26G crimped needle along the wall of the flask containing the cationic lipid solution so as to prevent the formation of foam. Gentle stirring takes place as soon as the two solutions have mixed. Finally a composition comprising 0.375 mM of DMRIE-DOPE and 500 μg/ml plasmid is obtained.

It is desirable for all the solutions used to be at ambient temperature for all the operations described hereinbefore. DNA/DMRIE-DOPE complexing takes place at ambient temperature for 30 minutes before immunizing the animals.

DNA vaccines can also be produced with DNA solutions containing plasmids pFC104 (example 6) and pFC106 (example 8) or containing plasmids pFC105 (example 7) and pFC106, plasmids pFC115 (example 17) and pFC106, or containing plasmid pFC101, pFC102 and pFC103 (examples 3 to 5), or containing plasmid pFC105 or pFC115 according to the procedure described in the present example.

### Example 21

In vitro Expression Tests

The expression of WN proteins is tested for each construction by conventional indirect immunofluorescence and Western Blot methods.

These tests are carried out on 96 well plates containing CHO cells cultured in monolayers and transfected by plasmids or containing CEF cells cultured in monolayers and infected by recombinant viruses.

The WN proteins are detected by the use of infected chicken or horse sera and of labelled anti-sera.

The size of the fragments obtained after migration on agarose gel is compared with those expected.

### Example 22

Effectiveness on Animals

The recombinant vaccines and plasmid vaccines are injected twice at approximately two week intervals into approximately seven day old, unvaccinated SPF chickens by the intramuscular route and in a volume of approximately 0.1 ml. An unvaccinated control group is included in the study.

The chickens are challenged by subcutaneous administration into the neck of 10^{3.4}TCID₅₀ of pathogenic WN virus.

Viremia, antibody response and mortality are observed. Autopsies are carried out to observe lesions.

### Example 23

Titrating Anti-WNV Neutralizing Antibodies

Dilution series are produced for each serum at a rate of 3 in DMEM medium to which was added 10% fetal calf serum in 96 well plates of the cellular culture type. To 0.05 ml of diluted serum is added 0.05 ml of culture medium containing approximately 100 CCIP₅₀/ml of WNV. This mixture is incubated for 2 hours at 37° C. in an oven in an atmosphere containing 5% CO2.

0.15 ml of a suspension of VERO cells containing approximately 100,000 cells/ml was then added to each mixture. The cytopathic effect (CPE) was observed by phase contrast microscopy after 4 to 5 days culturing at 37° C. in an atmosphere containing 5% CO₂. The neutralizing titers of each serum are calculated using the Kärber method. The titers are given in the form of the largest dilution inhibiting the cytopathic effect for 50% of the wells. The titers are expressed in log 10 VN50. Each serum is titrated at least twice and preferably four times.

### Example 24

Test on Horses of vCP2017

Recombinant vaccines containing vCP2017 (example 18) formulated extemporaneously with 1 ml of Carbopol© 974P adjuvant (4 mg/ml) were injected twice at 35 day intervals into horses aged more than three months and which had not been previously vaccinated, using the intramuscular route and a volume of approximately 1 ml. Three groups of animals were vaccinated, with doses of 10^{5.8}CCID₅₀ (i.e. 10^{5.64} pfu) for group 1, 10^{6.8}CCID₅₀ (i.e. 10^{6.64} pfu) for group 2 and 10^{7.8}CCID₅₀ (i.e. 10^{7.64} pfu) for group 3. An unvaccinated control group was included in the study.

The serology was observed. The neutralizing antibody titers were established and expressed in log10 VN50, as indicated in example 23.

| Group | Titers at day 0 | Titers at day 35 | Titers at day 49 |
|---|---|---|---|
| 1 | <0.6 | <0.78 | 2.66 |
| 2 | <0.6 | 1.14 | 2.58 |
| 3 | <0.6 | 1.16 | 2.26 |
| control | <0.6 | <0.6 | <0.6 |

Having thus described in detail preferred embodiments of the present invention, it is to be understood that the invention defined by the appended claims is not to be limited to particular details set forth in the above description as many apparent variations thereof are possible without departing from the spirit or scope of the present invention.

1-20. cancel

21. An immunogenic composition to induce an immune response against West Nile Virus (WNV) in an animal susceptible to WNV comprising a vector comprising a recombinant avipox virus or DNA plasmid that contains and expresses in vivo a nucleic acid molecule encoding WNV protein prM-M-E.

22. The immunogenic composition of claim 21, wherein the vector is the recombinant avipox virus.

23. The immunogenic composition of claim 22 wherein the avipox virus is a canarypox virus.

24. The immunogenic composition of claim 23 wherein the canarypox virus is an ALVAC canarypox virus.

25. The immunogenic composition of claim 21 wherein the vector is the DNA plasmid.

26. A method for inducing an immune response against WNV comprising administering to the animal the immunogenic composition of any one of claims 21-25.

27. The method of claim 26 wherein the animal is a horse.
